(19) United States
(12) Patent Application Publication
(10) Pub. No.: US 2015/0150726 A1
(43) Pub. Date: Jun. 4, 2015

(54) HEADGEAR TO REDUCE TRAVEL SICKNESS

(71) Applicant: Timothy Flaxman, Great Yarmouth (GB)

(72) Inventor: Timothy Flaxman, Great Yarmouth (GB)

(21) Appl. No.: 14/403,950

(22) PCT Filed: May 28, 2013
(86) PCT No.: PCT/GB2013/051411

§ 371 Date: Nov. 25, 2014

(30) Foreign Application Priority Data
May 25, 2012 (GB) 1209320.9

(51) Int. Cl. A61F 9/04; A42B 7/00; A42B 1/06

(52) U.S. Cl. CPC A61F 9/045; A42B 1/064; A42B 7/00

(57) ABSTRACT

A cap worn on the top of the wearer's head, having two flaps hinged under the peak along the axis allows each flap to be raised and temporarily fastened under the peak, alternately exposing each eye to clear vision or the restricted vision of an image of a flap that is sufficiently opaque to light to prevent the user from perceiving images through the flap.

### CROSS-REFERENCE TO RELATED APPLICATIONS

This application is a national phase entry under 35 U.S.C. §371 of International Patent Application PCT/GB2013/051411, filed May 28, 2013, designating the United States of America and published in English as International Patent Publication WO 2013/175245 A1 on Nov. 28, 2013, which claims the benefit under Article 8 of the Patent Cooperation Treaty and under 35 U.S.C. §119(e) to Great Britain Patent Application Serial No. 1209320.9, filed May 25, 2012, the disclosure of each of which is hereby incorporated herein in its entirety by this reference.

### TECHNICAL FIELD

The present invention relates to the technical field of a device to reduce or prevent feelings of travel sickness in a user. In particular, headgear, such as a cap, is provided for the user to wear during a journey.

### BACKGROUND

Travel sickness or kinetosis is a common condition that is typically caused by repeated lateral movements while travelling. So, for example, a person afflicted with this condition may not suffer the symptoms, nausea or vomiting, while travelling in a straight line but may suffer when the direction of travel changes at intervals, especially irregular or sudden intervals. The mode of transport causing the symptoms will vary between sufferers, but can be a car or a coach, a boat, airplane, or by going on certain fairground rides such as a rollercoaster or a merry-go-round. In severe cases, symptoms can persist for several hours following completion of a journey. In some cases, travel sickness can be brought on through watching moving images, particularly where the images show rapidly changing motion, on a television or at the cinema.

A number of remedies have been proposed to alleviate the symptoms or to remove them entirely. Medications are available, either over-the-counter or by prescription. For example, antihistamines such as Promethazine are used, but can cause significant drowsiness as a side effect. Also, alkaloids such as scopolamine are known to be used in the form of transdermal patches, but extreme care must be taken due to the side effects.

Non-chemical methods are also known, which can be as simple as sleeping throughout the journey or staying in the fresh air. Alternatively, sufferers from car or coach sickness are advised simply to look through the front windshield of the vehicle and to refrain from looking out through the side windows.

### BRIEF SUMMARY

It is an object of the present invention to provide a cap that will substantially reduce or prevent the wearer from suffering the effects due to travel sickness.

According to a first aspect of the invention, there is provided headgear comprising one or more flaps, each flap being attached to the headgear and deployable over an eye of a wearer, wherein each flap is sufficiently opaque to light to prevent the wearer from perceiving images through the flap when the flap is deployed.

Preferably, each flap is removably attachable to enable the headgear to be used as normal when a user is not travelling.

Advantageously, the duration of eye coverage by a flap is determined by the use of an integral electrical timer to give visual or audible indication of time elapsed. Further, advantageously, the timer is coupled to an electromechanical device to deploy or retract a flap. Optionally, the opacity of a flap is complete, but allows light to enter the eye around the periphery of the flap.

Conveniently, individual eye coverage is achieved by means of a vertically or horizontally sliding flap.

Preferably, individual eye coverage is achieved by means of one flap hinged in such a way that either eye can be covered by the flap.

Preferably, the headgear has two flaps so that both eyes can be covered by a flap at the same time to allow a user to better relax.

The headgear is preferably a peaked cap, each flap being attached beneath the peak of the cap so that a flap is close to the eye region. A flap can advantageously be raised and fastened to the underside of the peak such that the flap is minimally visible to onlookers to allow the cap to function as a normal cap.

According to a second aspect of the invention, a flap is provided, suitable for attachment to headgear, including a cap, which can be temporarily or permanently fixed to the headgear or cap.

### BRIEF DESCRIPTION OF THE DRAWINGS

The invention will now be described with reference to the accompanying drawings, which show by way of example only three embodiments of headwear. In the drawings:

FIG. 1 is a perspective view of a cap in accordance with a first embodiment of the invention.

FIG. 2 is a side view of a cap in accordance with a first embodiment of the invention.

FIG. 3 is a perspective view of a sun visor in accordance with a second embodiment of the invention.

FIG. 4 is a perspective view of a headband in accordance with a third embodiment of the invention.

FIGS. 5(a) and 5(b) are schematic side elevation views of a cap in accordance with a fourth embodiment of the invention.

FIG. 6 is a top view of attachable flaps in accordance with a fifth embodiment of the invention.

### DETAILED DESCRIPTION

In its basics, the invention provides headwear in the form of a cap or hat, or a sun visor or a simple headband, to be worn by a person who suffers from travel sickness, from which an attached flap or flaps hanging downward from the headwear, restrict the view of one eye to, at most, a peripheral image from around the edge of the flap. Normally, only one flap is used at a time, but by providing two flaps, the view of each eye, in turn, can be restricted for an equal duration, thereby mitigating fatigue.

Without being bound to theory, it is believed that sickness arises from a mismatch of the three-dimensional image formed in the brain, together with sensory impulses received from the balance mechanism within the ear. By removing the ability of the user to perceive a three-dimensional image in preventing the perception of movement in one eye, but still allowing observation of the user's surroundings in the other eye, the mismatch is tampered with or removed and the travel sickness resulting therefrom does not occur. Critically, the eye that has vision restricted should remain open and should receive some illumination. If the eye that has vision restricted is shut or has no illumination, then the brain seemingly disregards any signal coming from it, and travel sickness ensues. It is envisaged that in order to be most effective, the invention should be worn immediately prior to the journey and for the entire duration of the journey.

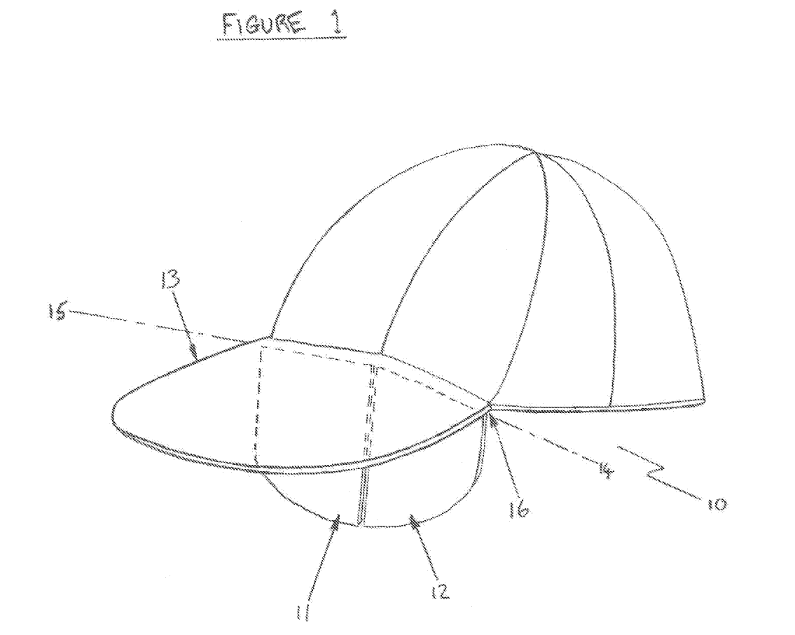

In FIG. 1, a baseball-type cap, generally referenced 10, is shown. Two flaps 11 and 12 made from fabric or other material, are shown hanging downward from the cap peak 13, and are individually hinged at axis lines 14 and 15. The flaps 11, 12 are separately movable between a vision-restricting position and a stowed position, where they are removed from restricting the visual field.

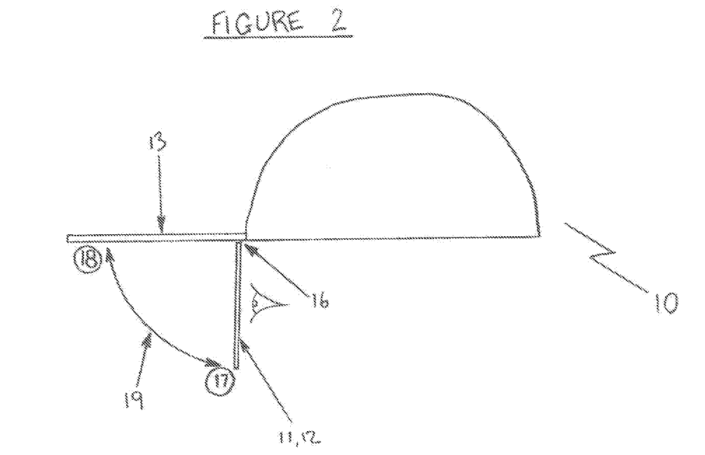

In FIG. 2, the baseball-type cap 10 is shown in side elevation to illustrate mechanical operation of the invention and the movement of flaps 11, 12 between the two positions. The arc 19, shows the limit of travel of the flaps 11, 12 from position 17, where flaps 11, 12 restrict the visual field of an eye, to position 18, where the flaps do not.

The flaps 11 and 12 are hinged starting at a point 16 and along the line 14, 15, enabling movement of the flaps 11, 12 to a position directly underneath and adjacent to the peak 13, to position 18, whereby they can be temporarily fastened and at which stage they no longer restrict vision.

The flaps 11, 12 are opaque to visible light to such an extent that the user cannot perceive an image through the flaps 11 and 12. Complete blockage of light by a flap 11, 12 is normally undesirable. It is, therefore, understood that although a flap can be fully opaque, there is no necessity for the flap to be completely so, only that insufficient light passes through to allow the eye and brain to discern individual objects and to perceive motion. When wearing the above cap 10, therefore, the user's uncovered eye, being unrestricted by the flap 11 or 12 that is fastened underneath the cap peak 13, receives a normal image therethrough but the eye behind the opaque flap only receives light from around the periphery of the cap 10. The user is thereby rendered less susceptible to travel sickness.

The flaps 11 and 12 can be secured to the cap peak 13 at position 18 by a number of means. First, a VELCRO® fixing would allow simple operation by the user to facilitate coverage of each eye throughout a journey to be of equal duration. If the cap has to be removed during a journey to enable alternating eye coverage, then travel sickness can occur quickly. In addition, fastening of the flaps 11 and 12 to the cap peak at position 18 can be achieved by pop-studs or buttons or magnetism.

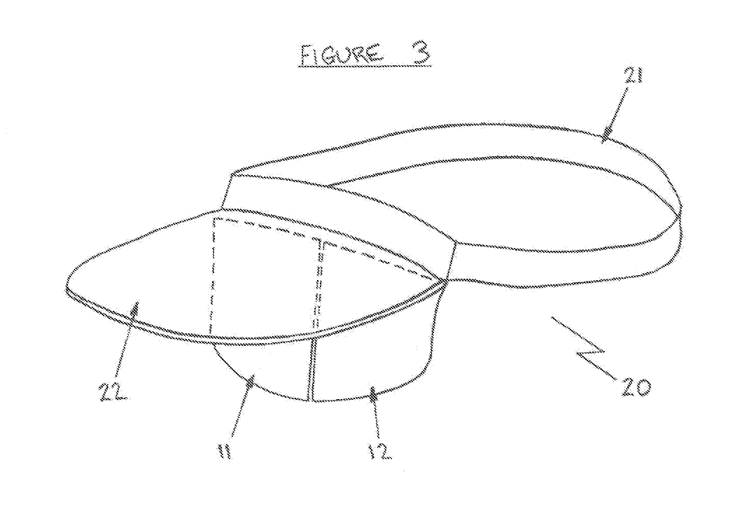

In FIG. 3, a sun visor, generally referenced 20, is shown as a further embodiment of the invention. In this example, a headband 21, is used to support the flaps 11 and 12 underneath visor 22.

A benefit of the invention is the ability to fasten both flaps underneath the visor 22 at the same time, allowing the user to employ the headwear for normal use. Moreover, a further benefit of the invention is the ability to allow both eyes to be covered at the same time to aid sleep or relaxation. In addition, decorations can be added to the flaps to, for example, increase the appeal of the headwear to children, a group that is particularly prone to travel sickness.

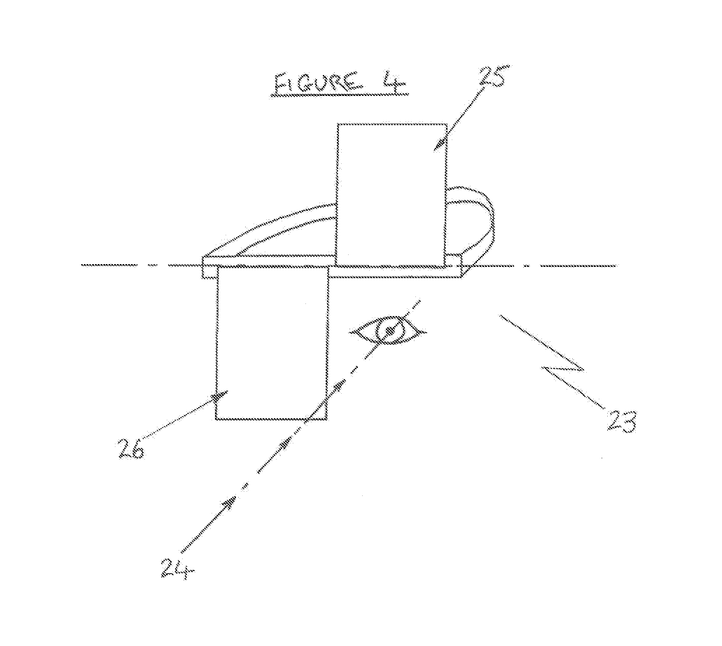

In FIG. 4, a headband, generally referenced 23, is shown demonstrating in this example, the line of sight 24 in the left eye to be clear of restriction by the flap 25 and the right eye covered by flap 26. In this embodiment, the flaps 25 and 26 can each slide vertically within slots (not illustrated) in the headband 23.

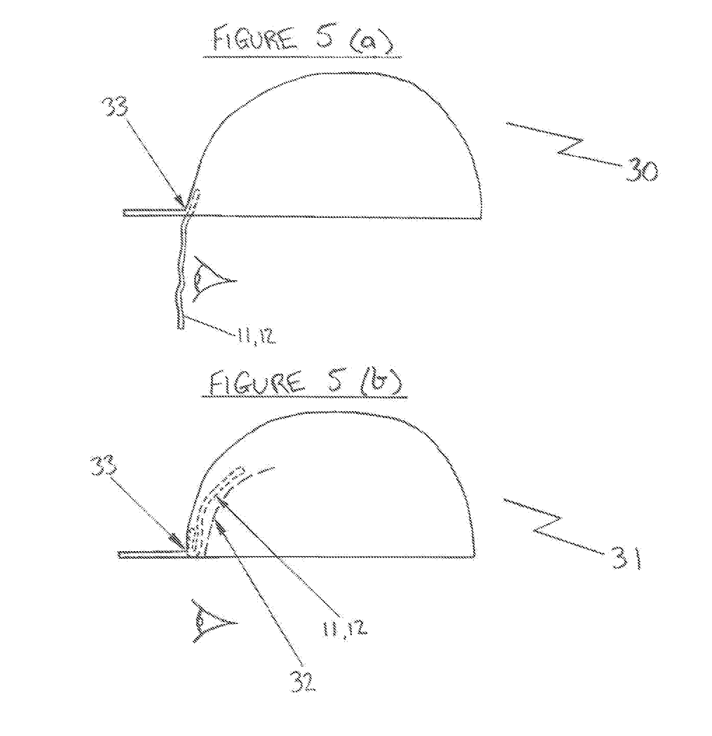

A further embodiment of the invention in FIG. 5(a) shows a cap generally referenced 30 having optionally removable flaps 11, 12 made of fabric or other material, temporarily attached to the cap at point 33, hanging downward and restricting vision. In FIG. 5(b), a cap, generally referenced 31, has the flaps 11, 12 optionally tucked inside the cap 31 resting on the user's forehead 32.

Optionally, the flaps are removably attached to the cap, enabling the cap to be used as normal when the user is not travelling. Moreover, in this embodiment, the flaps can be attached to any cap or headwear using temporary fastening methods such as VELCRO® or pop-studs or buttons or other temporary fastening methods.

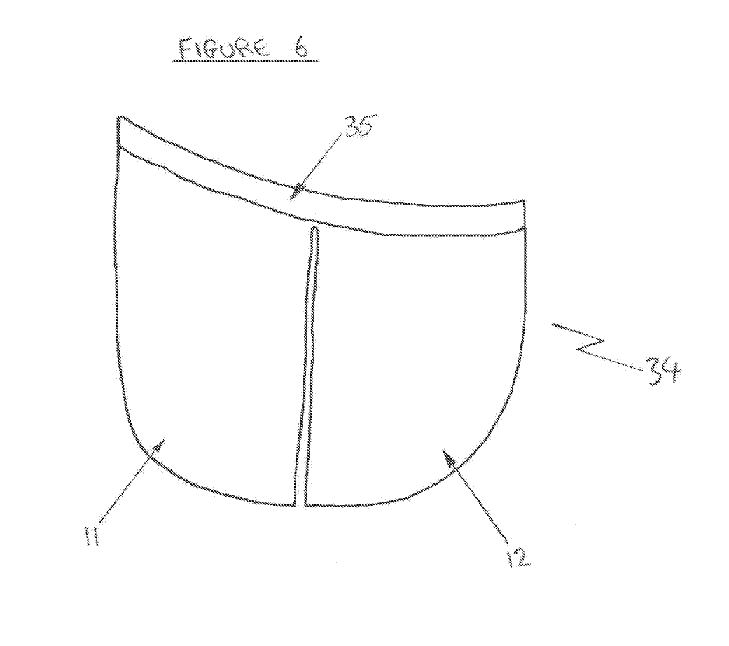

In FIG. 6, generally referenced 34, optionally attachable flaps 11, 12 are shown. The fastening area 35 can be optionally temporarily fastened inside the front rim of any suitable hat or headwear using VELCRO® or other suitable temporary fixing method.

In an additional embodiment, a pre-set duration of eye coverage can be achieved by the use of an integral electrical timer to give visual or audible indication of time elapsed with or without a coupled electromechanical device to deploy the flaps. In a further additional embodiment, one or more areas of a flap, for example, the in-use lower edge, can include an adhesive material or material that clings to a user's skin, to ensure that the flap remains close to the user's face and is not easily moved, for example, by air current. Other means of reducing the movement of a flap when in use, well known in the art, can also be included.

It will, of course, be understood that the invention is not limited to the specific details described herein, which are given by way of example only, and that various modifications and alterations are possible within the scope of the invention.

1. Headgear comprising one or more flaps, the or each flap being attached to the headgear and deployable over an eye of a wearer and characterized in that the or each flap is sufficiently opaque to light to prevent the wearer from perceiving images through said flap when said flap is deployed, said flap being so shaped to allow light to enter the eye around the periphery of said flap when deployed.

2. The headgear according to claim 1, wherein the or each flap is removably attachable.

3. The headgear according to claim 2, wherein the duration of eye coverage by a flap is determined by the use of an integral electrical timer to give visual or audible indication of time elapsed.

4. The headgear according to claim 3, wherein said timer is coupled to an electromechanical device to deploy or retract a flap.

5. The headgear according to claim 4, wherein a flap is completely opaque.

6. The headgear according to claim 5, wherein individual eye coverage is achieved by means of a vertically or horizontally sliding flap.

7. The headgear according to claim 6, wherein individual eye coverage is achieved by means of one flap hinged in such a way that either eye can be covered by the flap.

8. The headgear according to claim 6, having two flaps, whereby both eyes can be covered by the flaps at the same time.

9. The headgear according to claim 6, wherein the headgear is a peaked cap, the or each flap being attached beneath the peak of the cap.

10. The headgear according to claim 9, wherein a flap can be raised and fastened to the underside of the peak such that the flap is minimally visible to onlookers.

11. A cap or headgear substantially as herein described with reference to the accompanying drawings.

12. The headgear according to claim 1, wherein the duration of eye coverage by a flap is determined by the use of an integral electrical timer to give visual or audible indication of time elapsed.

13. The headgear according to claim 12, wherein said timer is coupled to an electromechanical device to deploy or retract a flap.

14. The headgear according to claim 1, wherein a flap is completely opaque.

15. The headgear according to claim 1, wherein individual eye coverage is achieved by means of a vertically or horizontally sliding flap.

16. The headgear according to claim 1, having two flaps, whereby both eyes can be covered by the flaps at the same time.

17. The headgear according to claim 1, wherein the headgear is a peaked cap, the or each flap being attached beneath the peak of the cap.
